(19) United States
(12) Patent Application Publication
(10) Pub. No.: US 2016/0367442 A1
(43) Pub. Date: Dec. 22, 2016

(54) ORAL DRUG DELIVERY DEVICE AND METHODS OF USING THE SAME

(71) Applicant: UNIVERSITY OF IOWA RESEARCH FOUNDATION, Iowa City, IA (US)

(72) Inventor: Max T. BAKER, Iowa City, IA (US)

(21) Appl. No.: 14/897,121

(22) PCT Filed: Jun. 26, 2014
(86) PCT No.: PCT/US14/44296

§ 371 Date: Dec. 9, 2015

Related U.S. Application Data
(60) Provisional application No. 61/839,595, filed on Jun. 26, 2013.

(51) Int. Cl. A61J 7/00; A61M 37/00; A61M 31/00; A61K 9/00

(52) U.S. Cl. CPC A61J 7/0053; A61K 9/006; A61M 37/0015; A61M 31/002; A61M 2037/0023; A61M 2210/0625; A61M 2209/088

(57) ABSTRACT

A device to deliver drugs via transmucosal absorption in the oral cavity and methods of administering same. The device of the present invention includes a drug-releasing element, where the drug-releasing element includes at least one drug and where the drug-releasing element is capable of delivering at least one drug to the oral cavity, and an external securing element.

### CROSS-REFERENCE TO RELATED APPLICATIONS

This application claims priority to U.S. Provisional Application No. 61/839,595, filed Jun. 26, 2013, the entire disclosure of which is incorporated by reference.

### FIELD OF THE INVENTION

The present invention relates to a device to deliver drugs to the oral cavity of a patient. In particular, the device allows for the placement and securing of a drug-releasing element in the buccal area of the oral cavity from which a drug is released. The drug released can exert an effect in the oral cavity or be absorbed into the bloodstream by buccal, sublingual, pulmonary, nasal and/or gastrointestinal routes to cause drug effects in the body. The device can be used to provide anticonvulsant effects in the central nervous system. Methods of administering the device are also included.

### BACKGROUND OF THE INVENTION

Epilepsy affects more than 50 million people worldwide. The outward effect of an epileptic seizure can be a wild thrashing movement (tonic-clonic, seizure). In some cases, the person does not convulse, but rather, simply loses body control and slumps to the ground. A seizure can last from a few seconds to status epilepticus, a continuous group of seizures that is often life-threatening without immediate intervention (seizures lasting 30 minutes or more). In status epilepticus rapid treatment is critical to a positive patient outcome.

The antiepileptics (also commonly known as anticonvulsant drugs) are a diverse group of pharmaceuticals used in the treatment of epileptic seizures. The goal of an antiepileptic is to prevent or suppress the rapid and excessive firing of neurons that start and propagate a seizure. If the administration of the antiepileptic does not occur until after the start of the seizure, the drug would prevent the spread of the seizure within the brain and offer protection against possible excitotoxic effects.

For example, a typical treatment for someone who is actively seizing from status epilepticus is the administration of lorazepam, which may be repeated if there is no effect after 10 minutes, if there is no effect after two doses, barbiturates or propofol may be employed, However, in this treatment, the administration is achieved via intravenous injection. As understood by those administering such treatment, such administration is time consuming. Indeed, before infusing the anticonvulsant drug, a tourniquet must be applied to the patient, a vein must be located, and the intravenous line must be tested. If the patient is convulsing, the intravenous infusion is difficult. Furthermore, there are a number of compounds that are highly effective as antiepileptics, but are not absorbed well orally or rapidly and are not suited to rapid seizure treatment. In particular, when taken orally, intestinal and hepatic enzymes affect the absorption of these substances.

While intramuscular (IM) anticonvulsant injections also can be used and can be administered more rapidly than intravenous injection, the current number of drugs that can be suitably absorbed IM are limited and, even more so limited with respect to antiepileptic drugs. Moreover, IM injections also require an autoinjector.

Nerve gas attacks also induce seizures. In particular, the acute central nervous system signs of exposure to nerve agents, such as organophosphates, are loss of consciousness, seizure activity, and apnea. Effects from nerve agent begin within seconds to several minutes after exposure. In particular, loss of consciousness and onset of seizure activity have occurred within a minute of exposure to nerve gas. There is no latent period or delay in onset from vapor exposure. Effects may continue to progress for a period of time, but maximal effects usually occur within minutes after exposure stops. While IM injections are relatively quick, and suitability limited to a few drugs, there is no easy way to determine whether a person has been injected in a mass attack.

In both of these scenarios, the patient/victim is typically rendered incapable of administering his/her own treatment. Even in situations where an epileptic patient who is alone or has no care provider is able to sense that he/she is about to have a seizure, the self-administration of an oral drug may not be effective because oral absorption takes longer for effective central nervous system blood levels of the drug to be achieved, in addition, if a seizure starts during the act of swallowing a pill or capsule, the pill or capsule may obstruct the airway and/or not reach the stomach.

Therapeutic compounds are available on the market in the form of tablets, sprays, and films that can be delivered via the buccal or sublingual mucosa. For example, nitroglycerin is used in emergency situations to induce arterial vasodilation in an effort to relieve the immediate symptoms of a heart attack. Drug delivery via the buccal and sublingual lining allows the drug to bypass the gastrointestinal tract and hepatic portal system, which increases the bioavailability of the drug that would otherwise undergo hepatic first-pass metabolism, and it also protects the drug from degradation due to pH effects and digestive enzymes. However, the delivery devices to date are limited to single agents and are most often in patch or film form.

The use of a patch or film is not ideal for a variety of reasons including the required insertion of fingers into the mouth to properly place the patch or film. In addition, the patch may become unattached and present a choking hazard as well as not allow absorption of the drug. Other forms of buccal delivery include a lollipop type delivery, such as the “fentanyl lollipop”, which also presents hazards to an unresponsive patient. For example, the patient can choke on the device because the device is not secured. Moreover, buccal administration of a drug in a solution by a conventional syringe is problematic because the drug may not be placed properly in the mouth, the drug may be swallowed, there is no prolonged release of drug and, once administered, there is not an opportunity to terminate drug dosing.

Accordingly, there is a need in the art for a device and a method of using the device to safely, effectively and rapidly administer drugs to the oral cavity where they may be absorbed by buccal, sublingual, nasal and pulmonary routes. Indeed, the incorporation of certain substances into the device that have beneficial central nervous system activities but are not absorbed well orally when swallowed would overcome the current limitations with regard to the use of such substances because transmucosal absorption in the oral cavity would bypass the intestinal enzymes and the liver and allow the drug substance to enter the central nervous system with a greater bioavailability. It would also be beneficial to administer treatment with a device that is readily identifiable at all periods of dosing. In addition, it would be beneficial to provide a device and method that allows nontoxic doses of a drug into the buccal cavity so that overdose is not an issue. Moreover, it would be beneficial to provide a device and method that allows rapid termination of drug dosing in the event that the care provider decides to alter the treatment.

### SUMMARY OF THE INVENTION

The present invention is directed to a drug delivery device including a drug-releasing element, where the drug-releasing element includes at least one antiepileptic substance, and where the drug-releasing element is capable of delivering at least one anti-epileptic substance via absorption in the oral cavity, and a securing element connected to the drug-releasing element. In one embodiment, the securing element is formed at least partially from a plastic, a composite material, or soft foam. In another embodiment, the drug-releasing element includes a combination of at least two anti-epileptic substances. In yet another embodiment, the securing element includes an additional securing aid where the securing aid is selected from the group consisting of ratchet-like features, serrations, suction cups, adhesive materials, a tether, and combinations thereof. Furthermore, according to one embodiment, the drug delivery device includes a syringe feature attached to the drug-releasing element. In an alternate embodiment, the drug delivery device further includes at least one magnet mounted onto the securing element. In another embodiment, the drug delivery device includes a voltage source capable of applying a voltage between the drug-releasing element and the securing element via an electrical conducting material. In this aspect, the drug-releasing element and the securing element are electrically polarized.

In another aspect, the present invention is directed to a drug delivery device including a securing element having a first portion, a second portion, and a third portion; a drug-releasing element connected to the first portion of the securing element, where the drug-releasing element includes at least one drug or drug-containing substance, and where the drug-releasing element is capable of releasing the drug or drug-containing substance in the oral cavity. In this aspect, the at least one drug-containing substance is in the form of a pill, tablet, or capsule. In another embodiment, the at least one drug-containing substance includes an outer rapid drug-releasing film and an inner slower drug-releasing matrix. In one embodiment, the at least one drug-containing substance includes at least one anti-epileptic substance. In yet another embodiment, the drug delivery device includes a hinge feature disposed between the first portion and the second portion of the securing element. Further, the drug delivery device includes a handle attached to the second portion of the securing element.

In yet another aspect, the present invention is directed to a drug delivery device including a securing element having a first portion, a transitional portion, and a third portion, where the first portion is positioned inside the oral cavity of a patient, the transitional portion traverses between the lips of the patient, and the third portion is positioned against the cheek of the patient; and a drug-releasing element connected to the first portion of the securing element, where the drug-releasing element includes at least one drug, and where the drug-releasing element further includes at least one microneedle capable of delivering the at least one drug through contact of the oral cavity. In this aspect, the at least one drug is coated on an outside surface of the microneedle. In the alternative, the at least one drug is positioned in an internal cavity of the microneedle. In one embodiment, the microneedle is made of non-dissolvable material. In another embodiment, the microneedle is made of dissolvable material. In another embodiment, the length of the microneedle is about 0.10 mm to about 0.75 mm. The drug delivery device may also include an identification element attached to the securing element. Further, according to one embodiment, the drug-releasing element is connected to the first portion of the securing element by a connecting segment.

Both the foregoing general description and the following detailed description are exemplary and explanatory only and provide an explanation of the invention as claimed. The accompanying drawings are incorporated in and constitute part of this specification, and are included to provide a further understanding of the invention; to illustrate several embodiments of the invention; and, with the description, explain the principles of the invention.

### BRIEF DESCRIPTION OF THE DRAWINGS

Further features and advantages of the invention may be ascertained from the following detailed description in connection with the drawings described below:

FIG. 1A illustrates one embodiment of the device of the present invention;

FIG. 1B illustrates the basic shape and dimensions of the device according to one embodiment;

FIG. 2A illustrates planar configurations of the device of the present invention;

FIG. 2B illustrates nonplanar configurations of the device of the present invention;

FIG. 3 illustrates the securing element of the device of the present invention according to one embodiment;

FIGS. 4A-4E illustrate different configurations of the device of the present invention;

FIGS. 5A-5E illustrate various embodiments of attachments used on the device of the present invention;

FIG. 5F illustrates a side view of the device of the present invention when inserted into a buccal cavity of a patient according to one embodiment of the invention;

FIGS. 6A-6F illustrate various securing aids of the device of the present invention;

FIG. 7 illustrates different embodiments with regard to the shape/design of the external portion of the securing element of the device of the present invention;

FIGS. 8A-8C illustrate various features of the securing element of the device of the present invention;

FIGS. 9A-9B illustrate a binge feature of the device of the present invention;

FIGS. 10A-10C illustrate a syringe feature of the device of the present invention;

FIG. 11 illustrates a hydraulic feature of the device of the present invention;

FIGS. 12A-12B illustrate an electromagnet feature of the device of the present invention;

FIG. 13 illustrates a voltage feature of the device of the present invention;

FIGS. 14A-14B illustrates a signal generator feature of the device of the present invention;

FIGS. 15A-15B illustrate different embodiments of the internal drug-releasing element of the present invention;

FIG. 16 illustrates another embodiment of the internal drug-releasing element of the present invention;

FIG. 17 illustrates another embodiment of the device of the present invention;

FIG. 18 illustrates yet another embodiment of the device of the present invention; and

FIG. 19 illustrates the area for insertion of the device of the present invention according to one embodiment of the invention.

### DETAILED DESCRIPTION OF THE INVENTION

The present invention relates to a device for the delivery and release of drugs to the buccal area of the mouth. The device of the present invention advantageously allows safe, rapid and effective placement of a drug-releasing element into the oral cavity of a patient for buccal drug release.

### Device

The device of the present invention includes an internal drug-releasing element connected to an external securing element. When applied to the patient treated, the internal element resides in the buccal area of the mouth (between the cheek and teeth/gums) and the external securing element contacts the outer cheek. A patient may include, but is not limited to, a human, a canine, and an animal. In one embodiment, the external element functions to hold the internal element in place. The internal drug-releasing element may contain one or more drugs which when released from the internal element provide a beneficial effect to the body. By the term, “drug” as used herein, it is meant any small or large molecular weight molecule or substance that is believed to be beneficial to the body. Such compounds may include regulated pharmaceuticals and compounds not regulated by national drug regulatory agencies such as herbal and nutritional substances.

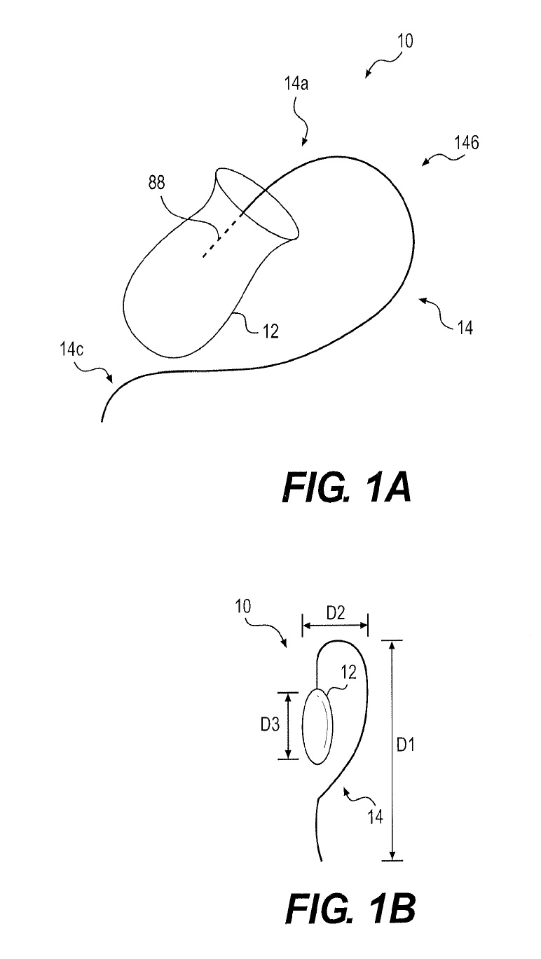

FIGS. 1A and 1B show the basic shape and dimensions of the device according to one embodiment. As shown in FIG. 1A, one embodiment of the device 10 includes an internal drug-releasing element 12 connected to a first portion 14a of the external securing element 14 by an optional connecting segment 88. The internal drug-releasing element 12 is the primary buccal contact area while the external securing element area 14c is the primary external cheek contact area. The first portion 14a of the securing element resides inside of the mouth when the device is in use. FIG. 1B shows the basic dimensions of the device according to one embodiment. As shown in FIG. 1B, the length of the device D1 is about 4 cm to about 10 cm, preferably about 6 cm to about 8 cm. The width of the device D2 is about 1 cm to about 3 cm, preferably about 1 cm to about 2 cm. The length of the internal drug-releasing element D3 is about 0.5 cm to about 5 cm, preferably about 1 cm to about 4 cm.

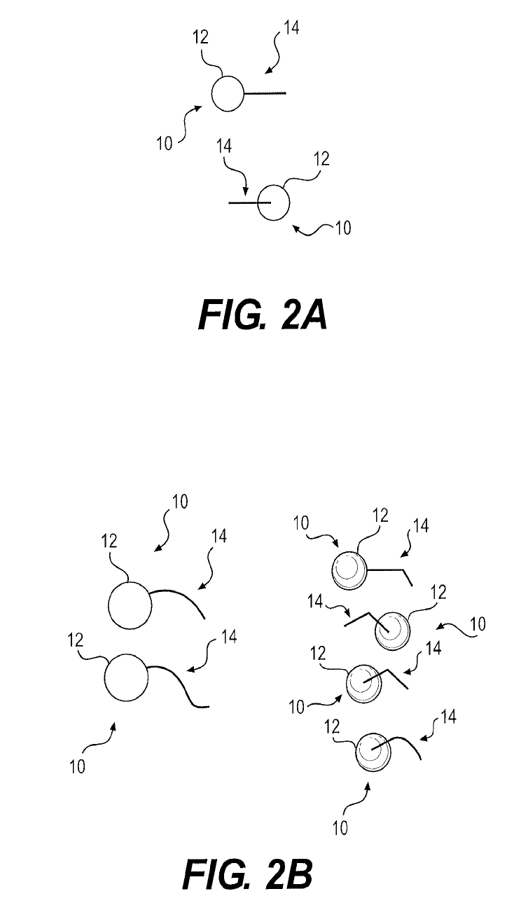

The external securing element 14 may be planar or nonplanar with the internal drug-releasing element 12. FIG. 2A shows views of a spherical or cylindrical internal drug-releasing element 12 and a planar external securing element 14. In a nonplanar configuration, as shown in FIG. 2B, the external securing element 14 may curve downward or upward at any point along the length of the external element. The external securing element 14 may be rigid or flexible, the latter of which will allow collapse inward so that the device 10 will not press in on the teeth of the patient when pressure is applied to the cheek from the outside. The angle may be a sharp angle or a smooth angle as depicted in FIG. 2B. The device 10 may also have other shapes that allow the external securing element 14 to maintain a stable spatial relationship with the internal drug-releasing element 12 in the buccal area.

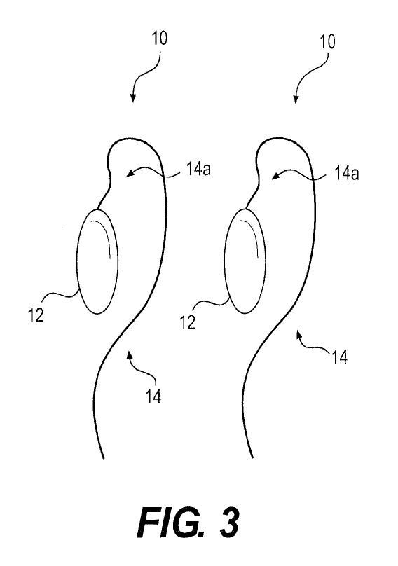

The first portion 14a of the external securing element 14 may be attached to an end of the drug-releasing element 12 via any suitable method that maintains a solid connection between the drug-releasing element 12 and the external securing element 14. In one embodiment, the securing element 14 is attached to the drug-releasing element 12 via an adhesive. In another embodiment, the first portion 14a is at least partially disposed within the drug-releasing element 12. In another embodiment, the first portion 14a may be indented as shown in FIG. 3.

The second portion 14b represents the transitional area that traverses between the lips. As shown in FIG. 1A, the transition may take the form of a continuous curved portion of the external securing element 14 that curves around the lip. In one embodiment, at least a portion of the front portion 14a may be flattened horizontally where it enters the mouth so that the patient can better form a seal between their lips and easily breathe through their nose. The external portion of securing element 14 (the portion between 14b and 14c) rests on the outer cheek and provides a closing force to keep the drug-releasing element 12 in place. However, in an alternate embodiment described in greater detail below, the transition portion may be a hinge-like portion that connects the internal portion of the securing element 14a to the external portion of the securing element 14b.

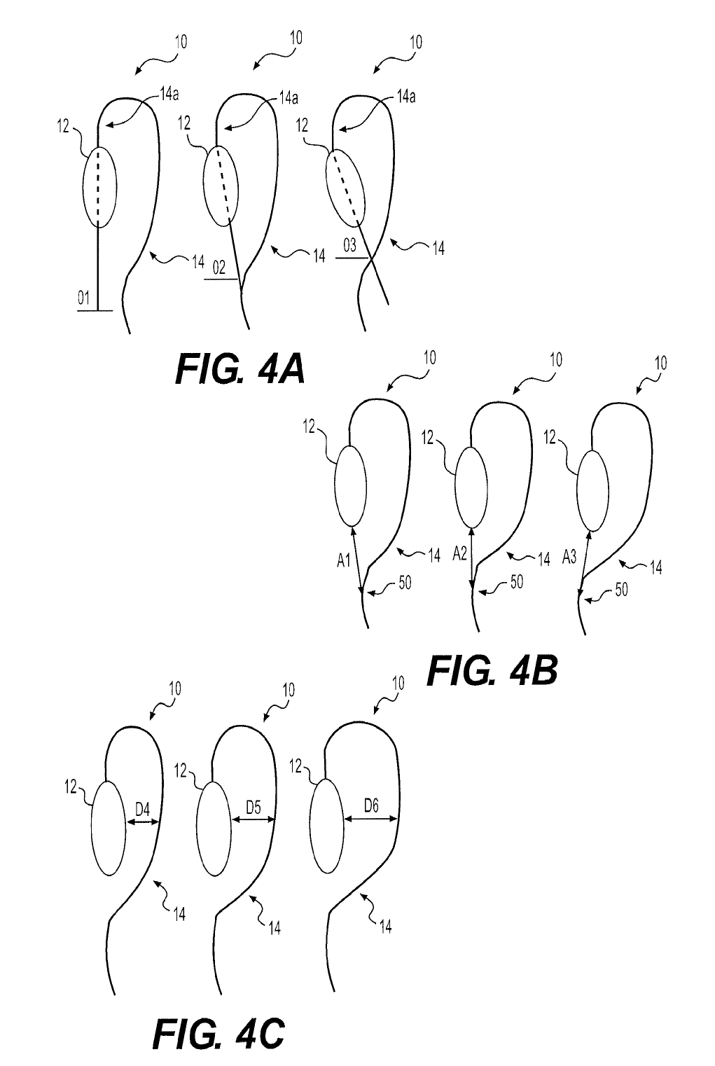

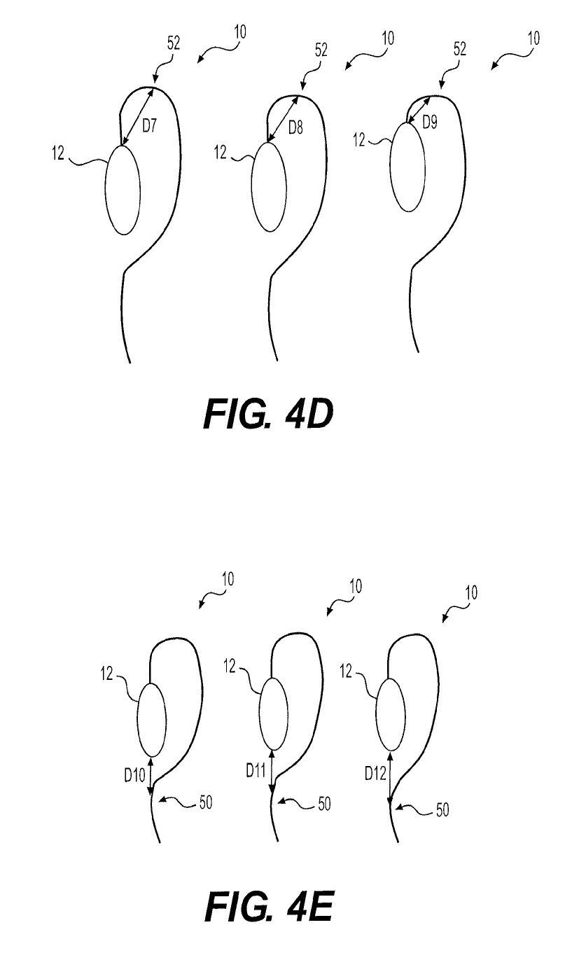

Different configurations of the device 10 are shown in FIGS. 4A-4E. FIG. 4A shows different angles of the internal drug-releasing element 12 relative to the first portion 14a of the external securing element 14. In one embodiment, the drug-releasing element 12 may be directly vertical to the first portion 14a. In another embodiment, the drug-releasing element 12 may form various angles θ1, θ2, θ3 with the first portion 14a. The drug-releasing element 12 may form an angle of about minus 10 degrees to about 30 degrees with the first portion 14a. FIG. 4B shows different alignments A1, A2, A3 of the drug-releasing element 12 with the external securing element 14 at the external securing element contacting point 50.

FIGS. 4C-4E show the distances between various aspects of the device. FIG. 4C demonstrates various lateral distances D4, D5, D6 between the drug-releasing element 12 and the securing element 14. In one embodiment, the lateral distance between the drug-releasing element 12 and the securing element 14 is about 0.75 cm to about 3 cm. FIG. 4D shows various distances D7, D8, D9 between the drug-releasing element 12 and the apex of the device 52. In one embodiment, the distance from the drug-releasing element 12 to the apex 52 is about 1 cm to about 4 cm. FIG. 4E displays various distances D10, D11, D12 between the drug-releasing element 12 and the external securing element contacting point 50. In one embodiment, the distance between the drug-releasing element 12 and the contacting point 50 is about 0.25 cm to about 3 cm.

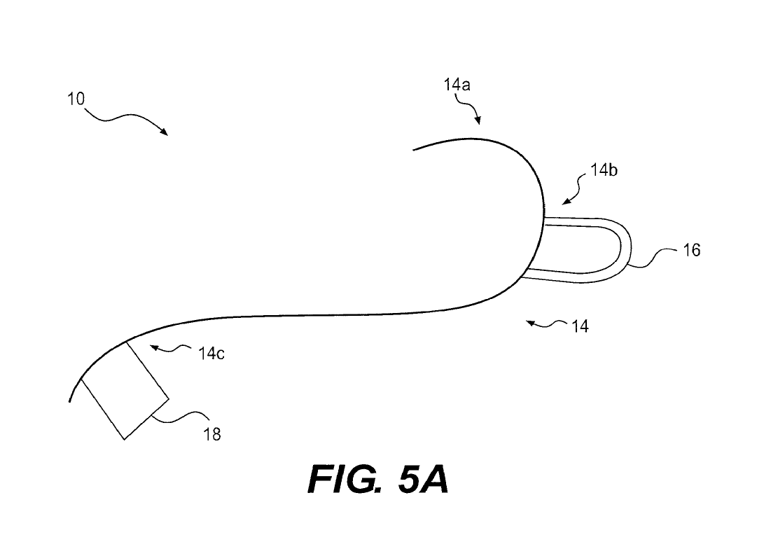

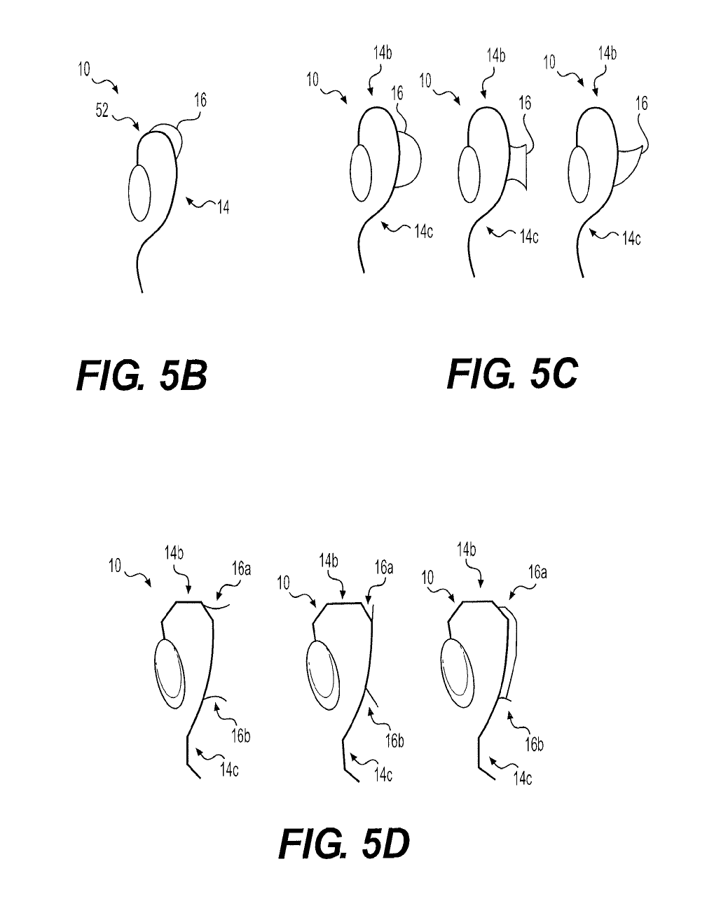

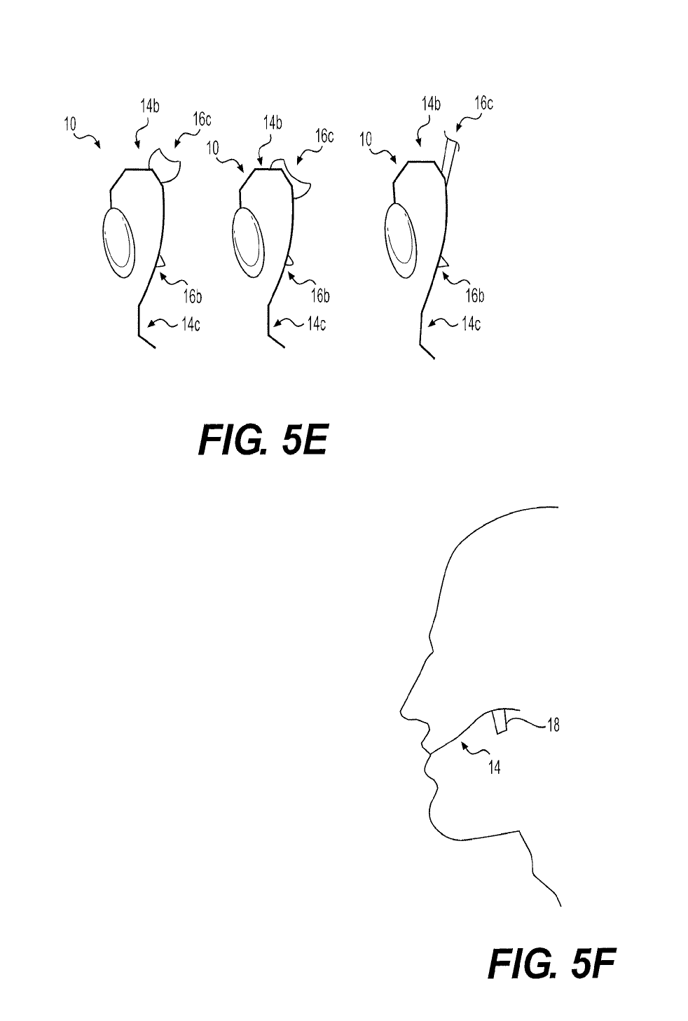

In one embodiment, as shown in FIG. 5A, the device 10 may also have an attachment on the second portion 14b of securing element 14 that functions as a handle or finger tab 16 to aid in the rapid and proper placement of the device 10. The attachment or tab 16 may be temporarily attached or permanently attached to the device. FIGS. 5B-5E show examples of tabs contemplated. En one embodiment, the device 10 includes a tab 16 at the apex 52 of the device as shown in FIG. 5B. In another embodiment, the device 10 includes a tab 16 on the external portion of securing element 14 (the portion between 14b and 14c) as shown in FIG. 5C. In yet another embodiment, the device 10 includes at least two tabs on the external securing element 14. For example, as shown in FIG. 5D, the device 10 includes an index finger tab 16a and a thumb tab 16b on the external portion of securing element 14 (the portion between 14b and 14c). By having both an index finger tab 16a and a thumb tab 16b, it is contemplated that the placement and removal of the device 10 will become easier. In an alternate embodiment, the device 10 includes a two-way tab 16c on the external portion of securing element 14 (the portion between 14b and 14c) as shown in FIG. 5E. The two-way tab 16c may be rectangular, for example a square rod, or the two-way tab 16c may be planar so that it lays flat. The two-way tab 16c may also have ridges on the flat surface to create a better grip. The two-way tab 16c may be used alone or in combination with the thumb tab 16b to insert and remove the device 10.

Likewise, the device 10 may have an identification element 18 (as shown in FIGS. 5A and 5F) that is attached to external portion of securing element 14 so that the time of administration, type of drug, and/or other pertinent details may be easily ascertained.

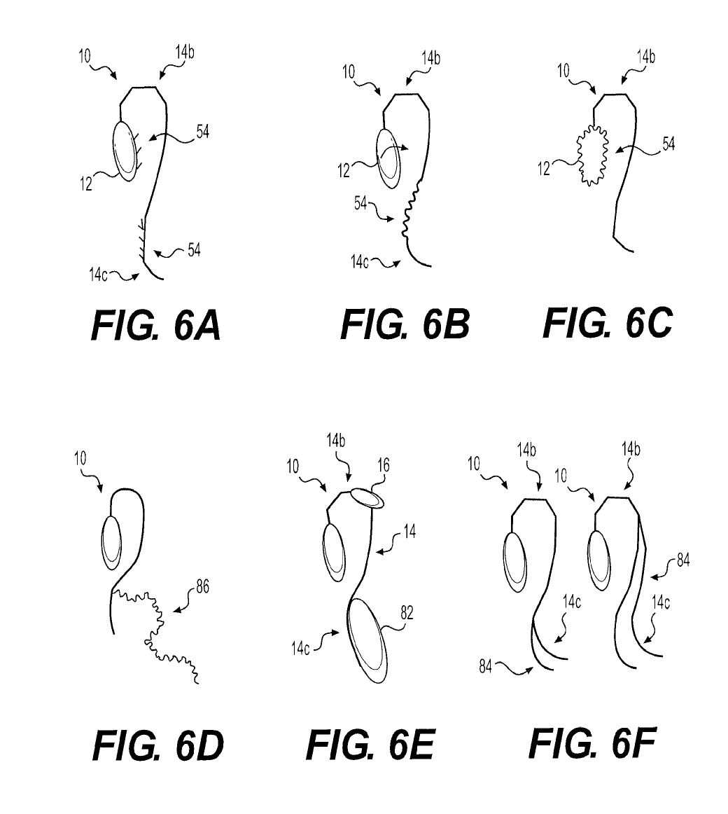

In another embodiment, the securing element 14 includes additional securing aids that allow further protection from displacement. For example, the external portion of securing element 14 anywhere between second portion 14b and third portion 14c may include ratchet-like features, serrations, suction cups, and adhesive materials such as bioadhesives, or combinations thereof that contact the outer cheek. FIGS. 6A-6C show various securing aids 54 used to inhibit the device 10 from backing out of the mouth. In another embodiment, as shown in FIG. 6D, the device 10 may be further secured with a necklace-like tether around the neck, a tether attached to the patient's clothing, or combinations thereof. For example, the tether 86 may be adhered to itself (e.g., tied around the patient's neck) with Velcro or a mechanical clip so that it will not tighten around the neck when tension is applied. In another embodiment, as shown in FIG. 6E, the external portion of the securing element 14 (the portion between 14b and 14c) includes an expansion point 82 to ease placement and deter misplacement. The expansion point 82 may be a flat disc, spooned shaped, flat loop, bent loop, sphere or any other suitable shape. In yet another embodiment, the external portion of the securing element includes a wye feature. As shown in FIG. 6F, the external portion (the portion between 14b and 14c) of the securing element 14 includes a wye feature 84 that provides greater stability and further aids in placement of the device. The wye feature 84 may also prevent rotation of the device.

The external securing element 14 may be formed of a variety of suitable materials providing the material achieves the overall goal of maintaining the internal drug-releasing element 12 in the buccal cavity. In one embodiment, the securing element 14 is formed of a wire. In another embodiment, the securing element 14 may be a wire coated with a thermoplastic. In still another embodiment, the securing element 14 is formed at least partially from a plastic or composite material. In yet another embodiment, the securing element 14 is formed at least partially from a malleable foam. The external securing element 14 may also be formed from any material that has a suitable resistance to movement and acceptable comfort against the cheek.

If there is a connecting segment 88 that connects the drug releasing element 12 to the securing element 14 and, more particularly 14a, this portion of the device may be made of metal, such as stainless steel, aluminum, platinum, or various metal alloys. In an alternate embodiment, this portion of the device may also be made of suitable plastics or composite materials including, but not limited to, pots/carbonate or carbon fiber materials.

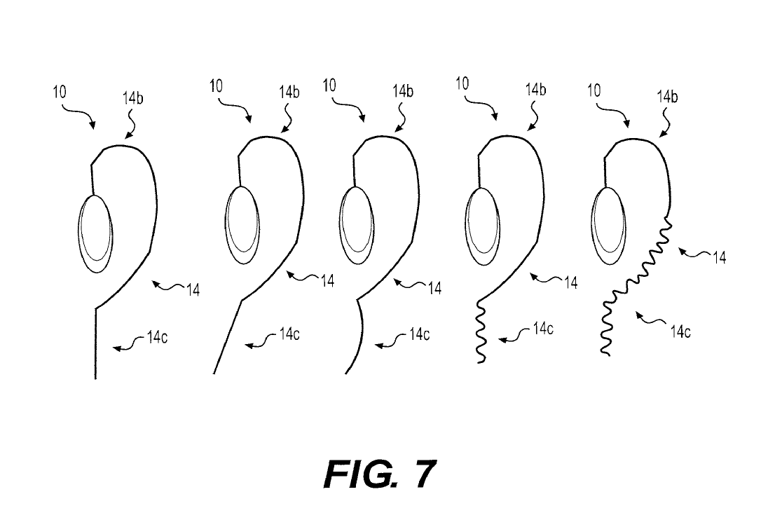

The shape of the securing element 14 may vary providing that the shape achieves the overall goal of maintaining the drug-releasing element 12 in the buccal cavity and holding the drug-releasing element 12 against the buccal membranes. For example, the securing element 14 is shaped such that it includes a third portion 14c that acts as a pressure point (primary cheek contact point) shown in FIGS. 1A and 5A so that, while in use, the device provides the correct amount and direction of pressure on the internal drug-releasing element 12 and the external securing element 14 to comfortably and properly maintain the location of the drug-releasing element 12 in the buccal area. Suitable shapes include, but are not limited to, spherical, cylindrical, square pad, ridged, or serrated. FIG. 7 shows different embodiments of the invention with regard to the shape design of the external portion (the portion between 14b and 14c) of the securing element 14.

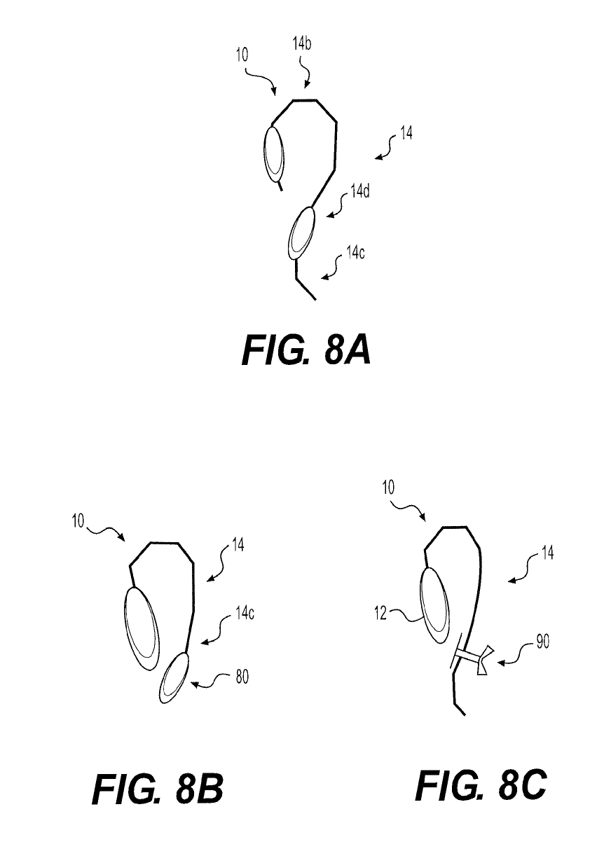

In another embodiment (generally shown in FIG. 8A), the external portion (the portion between 14b and 14c) of the securing element 14 has a moveable expansion feature 14d that can be moved along the external portion 14b so that the device can be adjusted for maximum comfort and stability for each patient. In another embodiment, as shown in FIG. 8B, the third portion 14c of the securing element 14 includes a gas or foam filled external balloon-like feature 80 to provide a comfortable, even pressure on the outer cheek. The external feature 80 may be sealed with a fixed gas content, or may be inflatable/deflatable with a valve and pumping system.

It is also contemplated that the device will have feature(s) that provide protection to the patient from external pressure on the device. Such features may be expanded features or additional physical features on the external element 14 that prevent direct pressure on the device 10 when the patient contacts a force to the area of cheek and mouth where the external element 14 is located. Such additional features may surround or cover the external element 14 and serve to absorb and/or disperse forces to other areas of the face. Such features may be connected to the external element 14 or not connected to the external element 14. They may be made of pre-formed foam or inflatable materials that may be inflated before or after application of the device 10. These features may have various shapes including spheroid, cylindrical, disk, or pillow shaped. The shape of the expanded feature may also conform to the outer cheek and mouth area. Furthermore, the internal drug-releasing element 12 and external securing element 14 may have inwardly collapsible features so that if a patient encounters pressure against it, such as in a fall or when their head rests on a pillow, the device will compress and lessen inward pressure to the mouth and teeth.

In still another embodiment, as shown in FIG. 8C, the device 10 may have a mechanism such as a screw type adjustor 90 to adjust the tension between the internal drug-releasing element 12 and the external securing element 14.

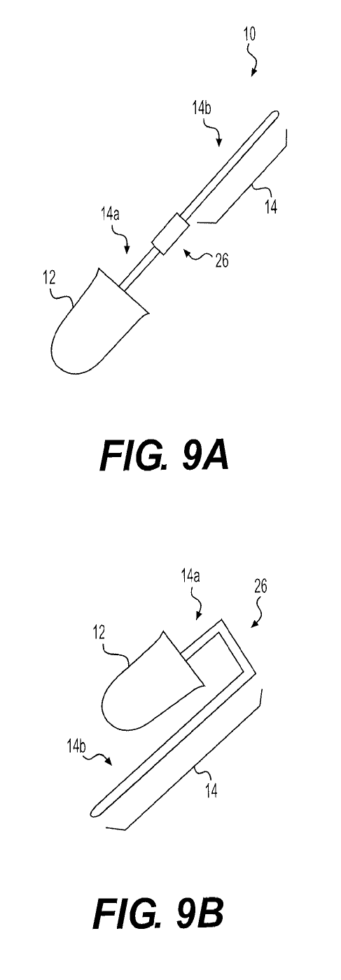

In another embodiment, the device may also have a hinge feature so that the drug-releasing element 12 can be placed between the cheek and teeth/gum when the whole device is elongated or in an extended or open form, but is closed and locked into place for drug delivery. For example, FIG. 9A shows a device 10 that includes internal drug-releasing element 12 and securing element 14 where securing element 14 has a hinge feature 26 disposed between the first portions 14a and second portion 14b of securing element 14. When the device 10 is in the open position, as depicted in FIG. 9A, the hinge feature 26 will allow for easier positioning of the drug-releasing element 12 in the buccal space by preventing insertion too far. FIG. 9B shows the device 10 when the hinge feature 26 is in the closed position.

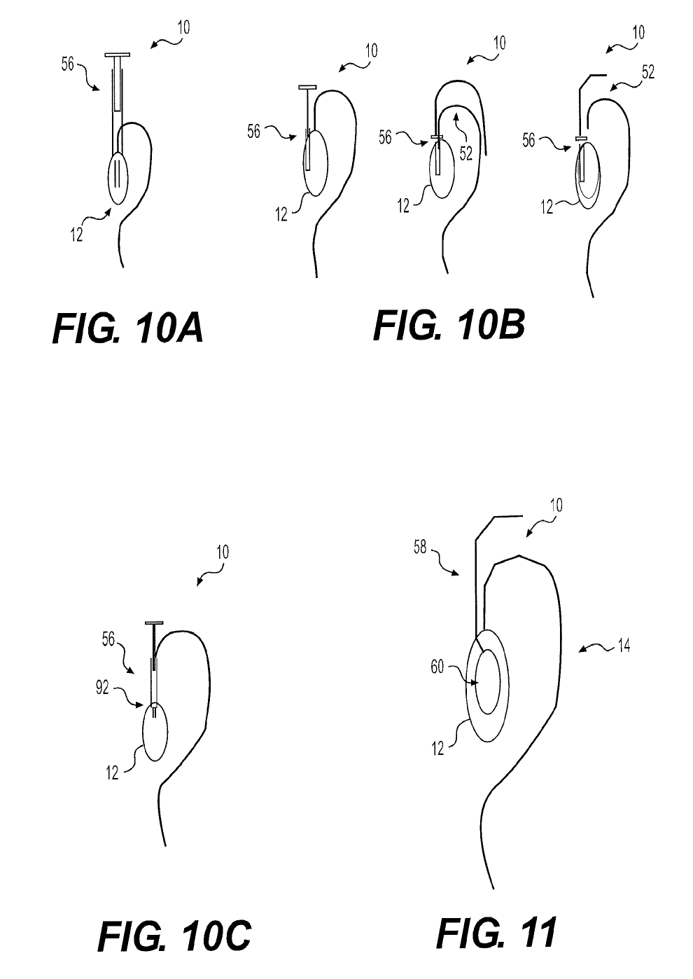

In an alternate embodiment, the drug-releasing element 12 has a syringe function (depicted in FIGS. 10A-10C). The syringe function 56 allows for storage and delivery of a liquid drug formulation. In some instances, a liquid-based drug formulation is preferred to achieve suitable drug dispersion. The device of the current invention allows for rapid, low risk placement of the syringe function that elutes a liquid drug formulation in the buccal space. It also allows for a controlled release of liquid drug formulation due to the presence of features to provide controlled release of the liquid and cause a delayed or prolonged drug release. Release of a liquid drug formulation that is too rapid can cause poor buccal drug absorption due to a portion of the liquid being swallowed. Also, rapid release of a liquid drug formulation does not provide prolonged drug effect in the CNS because there is no prolonged drug release.

In one embodiment, as shown in FIG. 10A, the syringe function 56 includes an absorbent material to slow the release of the drug once the syringe function 56 is depressed. FIG. 10B shows different designs of the syringe function 56. The syringe function 56 may be pressed or rotated to release the drug. In one embodiment, the syringe function 56 curves around the apex 52.

A syringe function will also allow for the rapid combining of a liquid substance with a solid substance. For example, a liquid acid or basic solution can be combined with a solid ionizable drug molecule to affect drug ionization, dissolution and/or absorption. Another example is that a liquid substance, such as water or saline, contacts a dry substance such as a bicarbonate salt (NaHCO₃) to create and release carbon dioxide (CO₂), an anticonvulsant gas. CO₂ released in the buccal space by this process will be inhaled by the patient. CO₂ released in this process can also facilitate drug dispersal by expanding the volume of a liquid drug solution due to the formation of CO₂ bubbles within the drug-containing liquid. In another embodiment, a liquid can be combined with a solid drug substance to create a drug solution which provides for drug dissolution and dispersal. This process is advantageous when a drug is not chemically stable in a liquid. For example, the benzodiazepine anticonvulsant drug lorazepam degrades over time in the presence of water. However, it is stable when in low water or in dry formulations. Therefore, the disclosed device provides a useful method for the buccal delivery of lorazepam and other drugs that are not stable in the presence of water.

In yet another embodiment, the internal drug-releasing element 12 is detachable from the syringe function. As shown in FIG. 10C, the connection between the syringe exit (what is commonly known as the “syringe tip”) and the internal drug-releasing element 12 can be made using a standard fitting 92 such as a Luer-Lock fitting. The tip of the syringe is the male end and the internal drug-releasing element 12 contains the female end. This detachability will allow for the exchanging of one internal drug-releasing element 12 for another. This may be desired if a faster or slower internal drug-releasing element 12 is desired for a specific treatment protocol. A faster or slower buccal internal drug-releasing element 12 may be varied as a function of the capacity and/or porosity of the internal element, for example.

A detachable internal drug-releasing element 12 will also allow for the syringe function 56 of the device (when the internal element is not attached) to be used to perform an intravenous injection. It can be made compatible with existing intravenous line ports by it having standard compatible Luer-Lock connections 92 (FIG. 10C). A detachable buccal internal drug-releasing element 12 may also be exchanged for an intranasal delivery element. This will allow the drug to be applied to the nasal cavity. In one example, an adapter component that creates a spray is applied to the syringe function 56 and administered by spraying into one or two nostrils of the patient. A needle may also be applied for an intravenous, intramuscular or subcutaneous injection. In these embodiments described, a detachable internal drug-releasing element 12 will allow health care providers to stock one drug containing item (the device having the drug formulation in the syringe function), but have the option to administer it in more than one way and thus increase utility. In another embodiment, the external securing element 14 of the device is detachable from the syringe function 56 so that the syringe portion can be used independently of the external securing element 14.

In another embodiment, as shown in in FIG. 11, the external securing element 14 has a mechanical or hydraulic connection 58 to a drug reservoir 60 in the internal drug-releasing element 12. The mechanical or hydraulic connection 58 can be used to cause spillage and/or forceful ejection of a drug formulation such as a spray of macro or microdroplets. Such a drug formulation may be released directly into the buccal space, or released into a buffering or moderating component, such as a porous material, to provide a controlled release. Portions of the drug formulation may be directly released and portions may be controlled released based on the capacity of the moderating (buffering) component. The drug may also be released so that it contacts a solid substance to cause a chemical reaction that facilitates drug absorption.

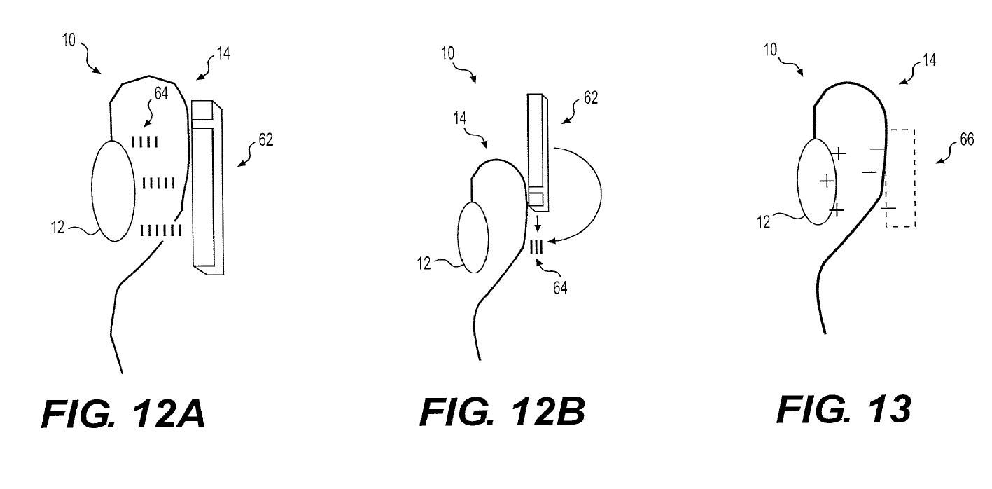

In another embodiment, the device has a permanent magnet or an electromagnet mounted on the external securing element. As shown in FIG. 12A, a permanent magnet or electromagnet 62 is attached to the external securing element 14 to exert a constant or oscillating magnetic field 64 to facilitate drug release and absorption. In one embodiment, the permanent magnet or electromagnet 62 may be activated by a button or switch. A magnetic force can be used to increase internal element immobilization due to the magnetic attraction of the internal drug-releasing element 12 to the external securing element 14. It can be used to increase the contact pressure between the internal drug-releasing element 12 and buccal membranes. It can further be used to facilitate drug release and drug absorption, by for example, causing the internal drug-releasing element 12 to vibrate due to an external oscillating magnetic field. In this embodiment, the internal element 12 will vibrate when it contains a magnetic responsive substance such as iron particles. In another embodiment, the magnetic force can be used to facilitate drug release and drug absorption by causing the directional movement of a ferromagnetic fluid containing a drug, or biocompatible iron-oxide nanoparticles containing a drug, from the internal drug-releasing element 12 to and through the buccal transmucosal membranes; and/or by causing a compression of a drug-containing compartment which forces the drug out of the internal drug-releasing element 12 into the oral cavity. The magnet may also perform these functions when placed in the internal drug-releasing element 12 rather than the external securing element 14. In this embodiment, as shown in FIG. 12B, the permanent magnet or electromagnet 62 may begin in an inactive configuration. However, the permanent magnet or electromagnet 62 may be moved by sliding or swinging to cause a magnetic effect 64 in the internal drug-releasing element 12.

In another embodiment, the internal drug-releasing element 12 or external securing element 14 may contain a heat generator, such as a battery powered heating element. When activated the heat will cause and/or facilitate drug release from the internal drug-releasing element 12. The heat being from about 37° C. to about 47° C., will also increase the absorption of drug by affecting physiological processes involved in drug absorption such as increasing vasodilation.

In an alternate embodiment, the internal drug-releasing element 12 and external securing element 14 are connected by an electrical conducting material such as a metal wire and a voltage is applied between the elements. As shown in FIG. 13, a voltage source 66, such as a battery, is applied between the internal drug-releasing element 12 and the external securing element 14 which are connected by a conductive material to cause each to act as an electrode. Polarity may be reversed to cause the internal drug-releasing element 12 to act as the cathode or anode. Such an electrical potential will cause a charged drug (a drug having a positive (+) or negative (−) charge), to move toward the electrical field having the opposite charge and away from the field having the same charge. For example, a drug having a positive charge such as midazolam in acidic solution will move toward an anode having a negative charge. This effect, known as iontophoresis, can be used to facilitate drug movement from the internal drug-releasing element 12 to and into the buccal tissue because the buccal tissue is located between the polarized internal drug releasing element 12 and external securing element 14.

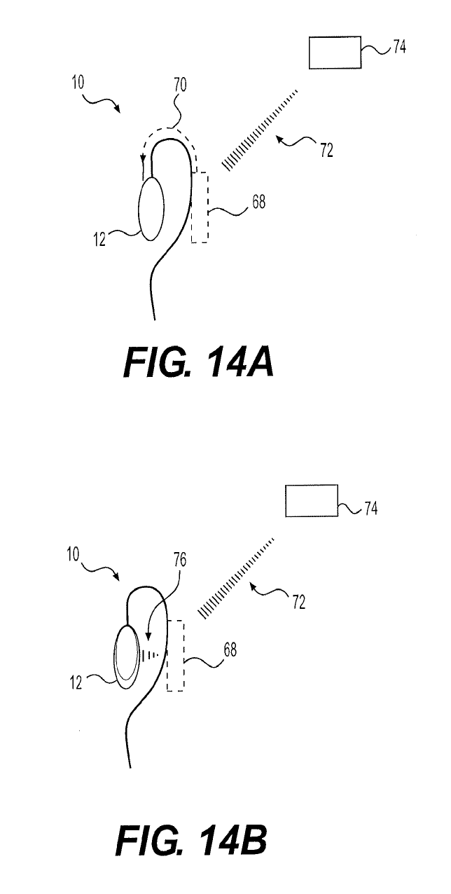

In one embodiment, the drug is released from the internal drug-releasing element 12 in response to an electromagnetic signal from a remote or wireless signal generator or from a signal generator attached to the external securing element. Such a signal generator may be an automated electronic seizure detector or a person such as a caregiver sending a radio signal. In this embodiment, as shown in FIG. 14A, the device 10 is equipped with an electromagnetic receiver 68 such as a radioreceiver which when activated at certain radiofrequencies sends an electrical signal 70 to the internal drug-releasing element 12 to cause release of drug. As shown in FIG. 14B, the electromagnetic receiver 68 may also send a wireless signal 76 to the internal drug-releasing element 12 rather than through an electrical connection. The radioreceiver 68 may be activated by receiving radiosignals 72 or other electromagnetic signals from a remote seizure detecting device 74 such as a motion sensor placed on the body of the patient or an EEG data collecting system which can detect seizures. The drug release process may be caused by the electrical signal 70 or wireless signal 76 to the internal drug-releasing element 12 causing the opening of a chamber containing the drug, or causing an electrical charge which releases the drug such as by iontophoresis, or causing heat which releases the drug through heat processes such as by causing a solid drug-containing matrix to become liquid. The electromagnetic receiver may also be placed in the internal drug-releasing element 12 rather than on the external securing element 14.

In another embodiment, a seizure detecting sensor is incorporated into the internal drug-releasing element 12 or external securing element 14 or both. Such a seizure detecting sensor can be made to detect seizures based on the patient's motor activities, i.e., movements of the head and head structures such as the jaw, or electroencephalographic (EEG) activities in the brain as detected by an EEG detecting component. Upon detecting a seizure, the internal drug-releasing element 12 is made to release the drug to treat the seizure.

According to another embodiment, the internal drug-releasing element 12 is equipped with microneedles that contact the buccal membranes and tissue. Microneedles may be from about 0.10 mm to about 1.0 mm, and preferably from about 0.20 mm to about 0.75 mm, in length. Microneedles will penetrate a short distance into and through the buccal membranes to facilitate drug uptake and delivery into the blood stream. Microneedles may be made to deliver a drug to the buccal tissue for uptake into the circulation by several methods. The drug may be coated on the outside surface of the microneedles, or the drug may be placed internally in cavities in the microneedles. When placed inside the microneedles, the drug may be expelled by diffusion or compressive forces from one or more channels in each microneedle. Microneedles may also be dissolvable when they contact the moisture of the saliva or tissue so that the drug, whether the drug is inside or outside the microneedles, will be released and dispersed upon microneedle dissolution. Dissolvable microneedles may be made of material that is solid when dry but becomes soft and dissolves when wet. Such materials include, but are not limited to, carbohydrates, dextrins, polysaccharides, sugars or poloxamer based material, maltose, carboxymethylcellulose, amylopectin, poly(methylvinylether/maleic anhydride), sodium hyaluronate, chondroitin sulphate/dextrin, sodium alginate, and hydroxypropyl cellulose. The use of the device having microneedles also allows for the administration of genetic therapy material such as RNA, DNA and live or inactivated viruses. In addition, therapeutic proteins such as antibodies and vaccines may be delivered with the device.

The internal drug-releasing element 12 includes a suitable delivery form of a drug(s), a therapeutic substance(s) or combinations thereof. A number of suitable delivery forms (drug-releasing substances) are contemplated for use in accordance with the present invention. For example, the delivery form may be a drug-containing matrix or drug-releasing substance, such as a container, pill, tablet or capsule. In one embodiment, the delivery form is a solid dosage form, such as a pill or tablet, or capsule affixed to the internal drug-releasing element. In another embodiment, the delivery form is a gel dosage form. In yet another embodiment, the delivery involves the release of drug-containing microspheres, macroemulsions, microemulsions, nanoemulsions, or nanoparticles. In another form, the drug-releasing substance contains an outer rapid drug-releasing film or coating and an inner slower drug releasing matrix.

In one embodiment, the delivery form of the drug-releasing element 12 is a non-dissolvable foam that is infused with at least one drug substance. For example, the drug-releasing element 12 may be made of non-aqueous soluble porous polymeric foam material. The drug(s)/substance(s) may be infused into the pores of the foam. When contacted by saliva, which acts as a solvent and carrier, the drug(s) will be washed out and released. The rate of release may be controlled in a variety of ways including adjusting the pore size.

In another embodiment, the internal drug-releasing element 12 contains material that dissolves in the oral cavity and causes drug release. In this aspect, the drug(s) substance(s) released may be complexed or bound to other substances to optimize absorption (e.g., cyclodextrins, polymeric materials such as poloxamers, pluronics or nanoparticles). The drug itself may be released unidirectionally, bidirectionally, or multi-directionally from the internal drug-releasing element 12. For example, the drug may be released only in the direction of the buccal membranes, or the drug may be released only in the direction of the teeth, or the drug may be released in all directions.

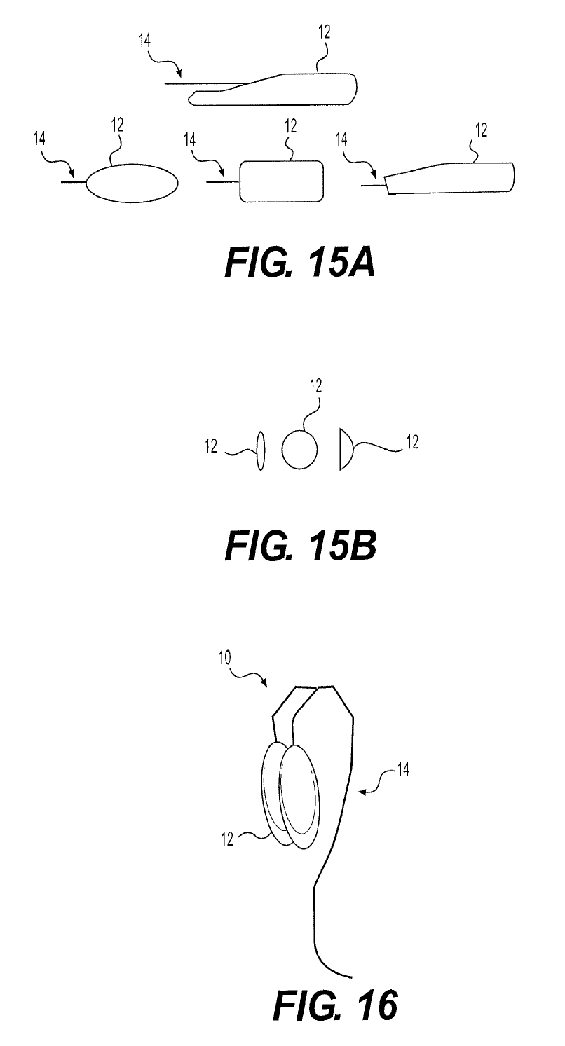

The internal drug-releasing element 12 may be cylindrical, rectangular, spherical, planar, or any suitable shape that allows the drug-releasing element to comfortably rest in the buccal cavity. FIG. 15A shows various shapes of the internal drug-releasing element 12. FIG. 15B displays an end view of the internal drug-releasing element 12. The surface of the internal drug-releasing element 12 may be smooth or irregular, such as dimpled (generally shown in FIG. 6C), to increase surface area and stability. En one embodiment, as depicted in FIG. 16, the internal drug-releasing element 12 may be compressible such that, when in use, it changes shape and conforms to the space in which it resides between the cheek and teeth. In another embodiment, the drug-releasing element 12 is composed of a bioadhesive material containing the drug.

The size of the internal drug-releasing element 12 may vary; however, it is preferably of a size that comfortably fits within the buccal cavity. In one embodiment, the internal drug-releasing element 12 may have a length of about 0.5 cm to about 5 cm, preferably about 1 cm to about 4 cm, and more preferably about 1 cm to about 3 cm.

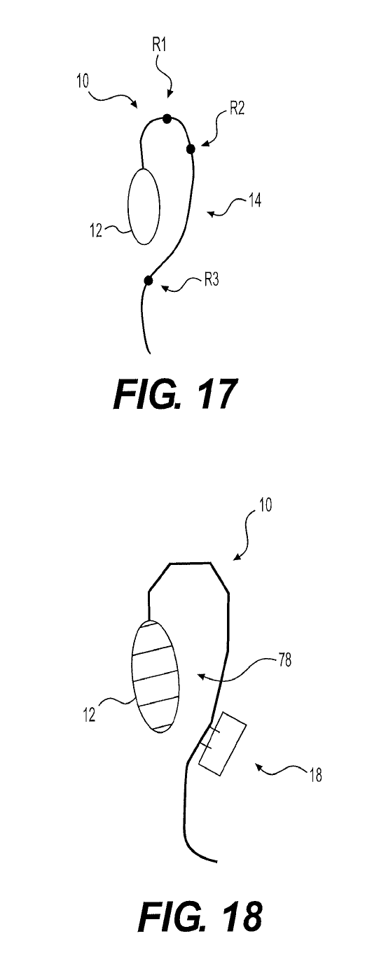

In another embodiment, the internal drug-releasing element 12 is rotatable on an internal shaft that is connected to or part of the first portion 14a of the external securing element 14. FIG. 17 shows various points of rotation R1, R2, R3 where the external securing element 14 may be twisted or rotated. The internal shaft may secure the internal drug-releasing element 12 with serrations, spines, or other physical features to cause the internal drug-releasing element 12 to adhere to the connecting segment 88. In yet another embodiment, the internal drug-releasing element 12 may be moveable along the connecting segment 88 so that the patient/administrator can better position the internal drug-releasing element 12.

There are a number of suitable substances that are contemplated for use with the above-described delivery forms. The substances delivered via the internal drug-releasing element 12 may be drugs/substances that are not effectively absorbed into the blood stream upon oral ingestion and/or drugs that are needed to rapidly enter the circulation. However, any substance can be delivered by the disclosed device. Drugs that are volatile can be delivered by the device. Drugs that are absorbed in the gastrointestinal tract (GI) can be delivered by the device by being released and swallowed by the subject.

In one embodiment, the substance delivered via the internal drug-releasing element 12 includes at least one highly lipophilic compound. A suitable example of a highly lipophilic compound contemplated for use with the present invention includes at least one anti-epileptic drug. In another embodiment, the device includes a combination of at least two anti-epileptic drugs. In yet another embodiment, the internal drug-releasing element 12 includes at least one herbal substance known to have anticonvulsant effects. For example, the drug-releasing element 12 may include carvacrol, thymol or the like. The drug-releasing element 12 may also include other substances capable of halting seizures such as diuretics, CO₂-releasing substances, and combinations thereof. Indeed, the device may also contain and deliver more than one antiepileptic or therapeutic substance in the same or different quantities.

A person of ordinary skill in the art would be aware of the effective amount of each drug/substance loaded into the internal drug-releasing element 12. For example, about 5 mg to about 700 mg of 2,6-disecbutylphenol is suitable for use in the drug-releasing element to effectively cause the cessation of seizures. Suitable examples of drugs/substances contemplated for use with the present invention include, but are not limited to: atropine, 2,6-diisopropylphenol, 2,6-di-secbutylphenol and its stereoisomers), 2-isopropyl-6-sec-butylphenol and its stereoisomers, 2-isopropyl-6-isopentyl phenol, acetazolamide (Diamox sequels), artemether, brivaracetam, bumetanide, carbamazepine (Carbatrol, Epitol, Equetrol, Tegretol), carisbamate, carvacrol, celecoxib, chlorodiazepoxide (Librium, Limbritrol, Librax), chlorothymol, clobazam (Onfi), clonazepam (Clonopin), corticosteroids/ACTH, curcumin, diazepam (Diastat, Valium), eserine, eslicarbazepine, ethosuximide (zarontin), ethotoin (Peganone), eugenol, eserine, ezogabine (Retigabine), felbamate (Felbatol), fenfluramine, fluorinated anesthetics (sevoflurane, isoflurane, methoxyflurane), fluorofelbamate, fosphenytoin, gabapentin (Neurontin), ganaxolone and other neuroactive steroids, huperzine, ICA-105665, ketamine, lacosamide (Vimpat), lamotrigine (Lamictal), levetiracetam (Keppra), lorazepam (Ativan), mephenytoin (Mesantoin), mephobarbital, methsuximide (Cleontin), midazolam, NAX-5055, oxcarbazepine (Trileptal, Oxtellar), paramethadione, pentobarbital, perampanel, perillyl alcohol, phenacemide, phenobarbital (Donnatal), phensuximide, phenytoin (Dilantin), PID, physostigmine, pralidoxime (2-Pam), pregabalin (Lyrica), primidone (Mysoline), progabide, propofol, rufinamide (Banzel), seletiracetam, somatostatin receptor agonists, STD (sec-butyl propyl carboxamide), stiripentol, sulthiame (Sulthiame), T-2000, T-2007, thiobarbital, tiagabine (Gabitril), tomatidine, topiramate (Topamax), trimethadione (Tridione), ursolic acid, valnoctamide, valproate (Depakene, divalproex (Stavzor), vapromide, VCD, vigabatrin (Sabril), YKP-3098, zonisamide (Zonegran), and combinations thereof.

Other substances that are suitable for use in accordance with the present invention include non-epileptic substances, analgesics, sedatives such as lipophilic imaging substances, vasodilating substances, surfactants or drug permeation enhancers. The latter group may include sodium lauryl sulfate, sucrose laurate, bile salts, Brij 35 or 98, Tween 20 or Tween 80, deoxycholate or various fatty acids. CO₂-releasing substances, acid substances, bronchodilating substances, antiemetic substances, and combinations thereof may also be administered with the device. Any of these substances may be used in combination with each other and in any combination with the anti-epileptic substances described above. Prodrugs of anticonvulsant compounds may also be administered.

For example, lipophilic non-drug substances, such as herbal compounds known to be beneficial to the CNS such as curcumin, resveratrol, carvacrol, peach extract, and the like, are contemplated for delivery with the device of the present invention. These substances may be administered for the treatment for patients with Alzheimer's disease and other CNS degenerative diseases. In particular, without being bound by any particular theory, carvacrol has been shown to have orofacial analgesic activity and to have antitumor effects and curcumin has been shown to have anticonvulsant effects. The device of the present invention may also be used to deliver the essential substances in rosemary oil, such as 1,8-cineole (eucalyptol), which is known to aid memory. The device of the present invention may also be used to administer other essential oils from various botanicals including lavender, peppermint, cherries, sage, and lemongrass.

Furthermore, lipophilic imaging substances, such as those used to image brain β-amyloid protein, e.g., ¹⁸F-florbetapir, are contemplated for delivery with the device of the present invention. Moreover, ursolic acid and tomatidine and their derivatives which have beneficial properties to the body and brain are contemplated for delivery with the device of the present invention.

It is contemplated that the device of the present invention be used to release an anticonvulsant gas into the oral cavity. The device will release the anticonvulsant gas CO₂ by it containing a bicarbonate salt (e.g., potassium or sodium bicarbonate), or other CO₂-releasing substances. CO₂ is an anticonvulsant gas known to inhibit seizures. Without being bound to any particular theory, it is believed that, upon insertion of the device and contact with the moisture, the CO₂-releasing substance will cause the patient to inhale CO₂ gas which will induce a rapid anticonvulsant effect. The drug-releasing element 12 may also include an acid substance such as ascorbic acid (vitamin C) or citric acid in combination with the CO₂-releasing substance to increase the rate of release of CO₂.

In this aspect of the invention, the rate of release of CO₂ may be controlled by the design of the drug-releasing element 12 so that the patient's inspired CO₂ (pCO₂) can be made to rise, but not rise high enough to cause hypoxia. In particular, since inhalation of about 5 percent CO₂ has been shown to result in the cessation of seizures, displacement of about 1-20 percent air with CO₂ gas is preferred.

In another embodiment, the drug-releasing element 12 may include the CO₂-releasing substance and at least one anti-epileptic substance. Without being bound to any particular theory, it is believed that the combination of at least one antiepileptic substance with a CO₂-releasing substance and, optionally an acid substance will produce a greater anticonvulsant effect. In particular, since CO₂ release from the device will cease over time due to the depletion of the CO₂-releasing substances, the device would be able to continue to release a longer acting anticonvulsant substance.

The device of the present invention is also contemplated for use in the administration of drugs to treat asthma. As such, the drug-releasing element 12 may include at least one anti-asthma drug. For example, since many drugs that can cause bronchodilation such as propofol and related phenols are not suitable for delivery as aerosols, the device of the present invention is believed to be an effective delivery device for lipophilic drugs that cause bronchodilation such that the drugs are allowed to enter the bloodstream and circulate to the pulmonary vasculature to cause bronchiolar relaxation. Vapors of the drug created by the drug's presence in the oral cavity may also be inhaled and directly contact the alveoli of the lungs to cause bronchodilation.

The internal drug-releasing element 12 may also include substances that treat nausea and vomiting. For example, antiemetic substances that are poorly absorbed into the circulation when taken orally are contemplated for use in the device of the present invention. Examples of such compounds include, but are not limited to, propofol, 2,6-sec-butylphenol, any compound classified as a cannabinoid, ondansetron, and combinations thereof.

The device may also be used to deliver drugs for the treatment of headache such as migraine headaches. Such drugs may be in the triptan class such as sumatriptan.

The device of the present invention is also contemplated for use on a patient with a toothache or dry socket pain such that at least one of the substances in the drug-releasing element 12 is an analgesic, a sedative, or a combination thereof. The device allows placement of the drug-releasing element 12 in the immediate vicinity of the aching tooth or dry socket such that the substance(s) is also released in the immediate vicinity of the aching tooth or dry socket, in one embodiment, the drug-releasing element 12 includes eugenol or other similar non-toxic phenol compounds to lessen pain. Other local anesthetics may be administered alone or in combination with eugenol for dental pain. Local anesthetics examples include lidocaine, procaine, tetracaine, and bupivacaine.

Moreover, sedatives and analgesics may be delivered with the device of the present invention. Examples of suitable sedatives contemplated for inclusion in the drug-releasing element 12 include, but are not limited to, midazolam, diazepam, lorazepam, dexmedetomidine, propofol, and combinations thereof.

The internal drug-releasing element 12 may also include antiseptic substances to inhibit microbe growth or kill microbes in the oral cavity. In addition, it is contemplated that the device will be a single-use product and, thus, an antiseptic will not be required for patient safety. In another embodiment, it is contemplated that the device or parts of the device will be re-usable and, thus, an antiseptic may be required.

In another embodiment, the ding-releasing element 12 may include preservatives and/or antioxidants, especially since a number of the lipophilic compounds discussed above are subject to oxidation and the inclusion of an antioxidant, preservative or other similar compound will act to preserve or protect the active drug substance until use. Non-limiting examples of suitable preservatives and/or antioxidants include benzyl alcohol, EDTA, ascorbic acid, citric acid, and combinations thereof.

The drug-releasing element 12 may also contain a lubricant to facilitate placement of the device. In addition, the drug-releasing element 12 may include menthols, sweeteners or other masking agents intended to mask the taste and odor of the drug(s) substance(s).

In one embodiment, the rate of release of the substance(s) in the drug-releasing element 12 may be controlled by modifying the design of the element so that it will release the substance(s) at predetermined rates. For example, seizures, particularly those from status epilepticus, may return after being stopped by a single dose of an antiepileptic drug because the blood/CNS levels of the antiepileptic drop below a therapeutic level. Accordingly, it is envisioned that the device of the present invention and, more particular, the internal drug releasing element 12 is capable of minimizing the drop in therapeutic blood levels by providing prolonged release of the drug into the oral cavity. Without being bound by any particular theory, it is believed that, because the drug-releasing element 12 is secured into place, substance release will be relatively consistent between patients.

In another embodiment, as shown in FIG. 18, the drug-releasing element 12 includes a plurality of segments 78 similar to slices or discs so as to allow each segment to contain a different substance so that dosing with more than one drug or substance can be achieved. For example, the drug-releasing element 12 may include more than one antiepileptic therapeutic substance or other substances in similar or different concentrations in each segment so that when placed in the buccal cavity, multiple substances are released. Any of the drug, non-drug, and other substances discussed above are suitable for use with the internal drug-releasing element 12 according to this aspect of the invention providing the combination of such substances does not counteract one of the substances or have an adverse reaction in the presence of another one of the substances used in one of the segments.

In still another embodiment, the device may include more than one internal drug-releasing element 12. For example, the device may include a drug-releasing element 12 for each buccal cavity where the drug-releasing elements are secured with a single external securing element 14 that passes around the patient's neck.

In yet another embodiment, the internal drug-releasing element 12 itself does not contain a CO₂-releasing substance. Rather, the external securing element 14 has a reservoir that contains CO₂-releasing substances. In such an embodiment, the CO₂ may be released by contacting the CO₂-releasing substance with water contained in an additional reservoir attached to an exterior portion of the device or kept completely separate from the device. In this aspect, the released CO₂ would travel into the oral cavity by a tube incorporated into the device. In another embodiment the external reservoir contains an anticonvulsant gas such as xenon (Xe) and/or CO₂. Such a reservoir may be a gas filled expandable element such as a balloon. Upon insertion, the gas is released by a triggering mechanism.

In another embodiment, the device of the present invention will release a volatile anesthetic such as isoflurane, enflurane, halothane, desflurane or sevoflurane. Volatile anesthetics are useful for stopping seizures and causing sedation.

The device of the present invention may also be used to absorb substances present in the oral cavity. According to this embodiment, the internal element 12 will be made of an absorbent material so that substances in the saliva or in exhaled breath will be taken up and absorbed into or onto the internal element. It is known that drugs pass from the blood through buccal and other oral membranes and into the saliva. Substances may be analyzed by removing the substance from the internal element 12, such as by extraction, and analyzed by methods such as gas chromatography, liquid chromatography, spectrophotometry, mass spectrometry or other suitable methods of analysis. In another embodiment, the substance will be absorbed into and analyzed by chemical reactions or sensors located in the internal element 12. For example, the internal element 12 can be made to undergo a color change when contacted with substances of interest, such as an illegal narcotic or substance of abuse. In this embodiment, the presence of the substance of interest is determined by visualization of the internal element 12.

Without being bound by any particular theory, it is envisioned that by having a substance-absorbing element in the oral cavity in contact with the saliva and buccal membranes for extended time periods, such as up to 30 min or 1 hour or more, the disclosed device will provide an accurate analysis of the substances of interest which may be present in low concentrations in the body and not conducive to analysis from a single saliva sample. In one embodiment, the device of the present invention is made with absorbing materials that have high specific affinities for the substance of interest. Such a device is advantageous because no blood samples are taken to measure the compounds of interest and it causes no discomfort or anxiety to the subject. Furthermore, a person having little or no skill in professional healthcare can administer the device or a subject can self-administer the device in any locale whether in or away from professional care.

In this aspect, the device of the present invention provides many benefits. For example, it may be desired to know if a therapeutic concentration of a drug is achieved in a patient. Another aim may be to determine if a subject is abusing drugs such as opioids, cocaine, cannabinoids or other drugs of abuse. In such cases, these compounds and/or their metabolites can be monitored with the device. In another aim, biomarkers of disease may be analyzed. Biomarkers are frequently present in low quantity requiring expensive precision instrumentation to measure. They may be volatile or nonvolatile. Because the device according to one embodiment of the present invention can absorb substances over extended periods, the device can concentrate the substance and allow a cheaper analysis. Moreover, genetic material or other cellular material, including RNA, DNA, and proteins may be absorbed and analyzed. This may be done for example, to determine the genetic makeup, the presence of specific mutations or the expression of certain proteins in the subject.

### Methods of Use

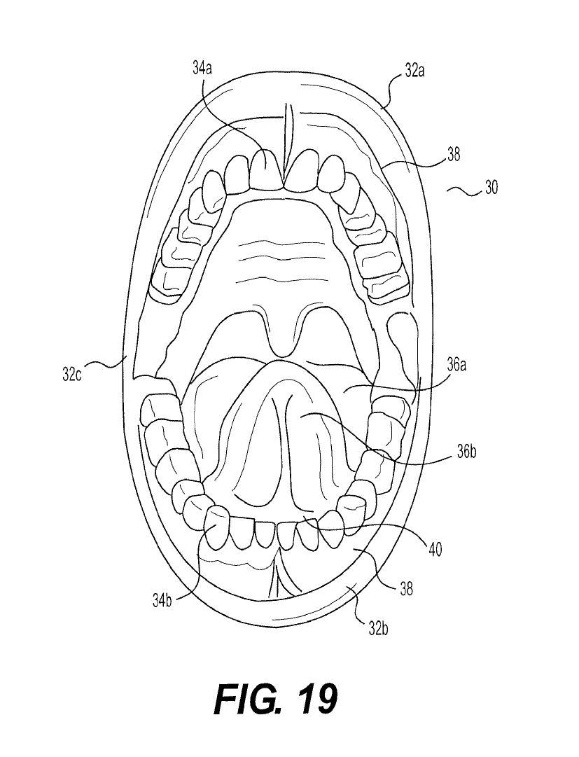

The device of the present invention may be applied rapidly into the buccal cavity, which is also referred to as the vestibule of the mouth. As shown in FIG. 19, the buccal cavity 38 is the area between the cheek 30 and the gums. Since the buccal cavity 38 is outside of the teeth 34a and 34b, the device of the present invention may be administered even if the patient is uncooperative, unresponsive, or has clenched teeth. Furthermore, since the securing element 14 is minimal, the patient's lips (32a, 32b, and 32c) may be allowed to close after administration without affecting the delivery of the substance.

The first portion 14a of the external securing element 14 preferably loops around the lip portion 32c. In the embodiment shown in FIGS. 9A-9B, the hinge 26 may rest against the lip portion 32c. As shown in FIG. 5F, the exterior portion of securing element 14 rests against the cheek.

The administration of the device of the present invention is preferably performed in about 30 seconds or less. In one embodiment, the insertion of the device in the buccal cavity occurs in about 20 seconds or less. In another embodiment, the insertion of the device in the buccal cavity occurs in about 10 seconds or less.

The release of the substance(s) in the internal drug-releasing element 12 occurs upon contact with the buccal membranes. In particular, when the device is in place, the drug or drug-containing substance(s) in the drug-releasing element 12 exits by diffusion, osmotic, compressive, electrical and/or magnetic forces. Without being bound to any particular theory, since the mucosa of the oral cavity, including the buccal region (the cheek), is highly vascularized, the substance(s) in the drug-releasing element 12 is taken up into the bloodstream and transported to the CNS (brain and spinal cord). Accordingly, the device of the present invention allows a patient to rapidly self-administer a drug or allows a care-provider to rapidly administer a drug to a patient or victim. In fact, since the drug-releasing element 12 is secured into place with the external securing element 14, no more action is required of the patient or care provider after application for the substance to be absorbed into the bloodstream. Furthermore, as discussed above, prolonged release and delivery of the substance(s) may be provided by design of the drug-releasing element 12.

This invention also has the advantage of allowing rapid termination of drug dosing. For example, if it is believed that too much of the drug is being absorbed, or if the care provider decides to switch treatments, the device can be immediately removed at any time by anyone with any skill level.

Another advantage of this device is that one care provider can rapidly treat many patients such as in a mass nerve gas attack. A single care provider can quickly insert the device in one patient and rapidly move on to the next. Moreover, the incorporation of an identification element 18 as shown and described in relation to FIGS. 5A and 5F allows for ease of identification of the patients/victims that have been administered drugs and other pertinent information.

In this aspect, the device of the present invention may be supplied in a sealed, rapidly accessible package. For example, the device may be supplied in a package that is quickly accessible to the user but maintains sterility of the contents until the package is opened for use. The package may be rigid, non-rigid, or contain elements of both. In one embodiment, the package is a disposable peel pouch. The peel pouch may be composed of paper/plastic combinations, Tyvek®/plastic combinations, or other suitable materials useful for peel pouches. The peel pouch may be sealed by heat-sealing the open end or by using a self-sealing pouch with an adhesive strip that is folded over the opening of the pouch.

Although the present invention has been described with reference to particular embodiments, examples, and drawings, it will be understood to those skilled in the art that the disclosure is exemplary only and that various other alternatives, adaptations, and modifications may be made within the scope and spirit of the present invention. For example, although the following discussion addresses exemplary configurations of the substance(s) to be included the drug-releasing element, the inventor contemplates other drugs and homeopathic compositions would also be useful. Furthermore, since anticonvulsants are also increasingly being used in the treatment of bipolar disorder and for the treatment of neuropathic pain, it is contemplated that the device and methods of the invention will be useful in the treatment of bipolar disorder, neuropathic pain, and similar conditions.

To the extent necessary to understand or complete the disclosure of the present invention, all publications, patents, and patent applications mentioned herein are expressly incorporated by reference to the same extent as though each were individually so incorporated. In addition, ranges expressed in the disclosure are considered to include the endpoints of each range, all values in between the end points, and all intermediate ranges subsumed by the end points.

What is claimed is:

1. A drug delivery device comprising:
a drug-releasing element comprising at least one anti-epileptic substance, wherein the drug-releasing element is capable of delivering the at least one anti-epileptic substance in the oral cavity; and
a securing element connected to the drug-releasing element.

2. The drug delivery device of claim 1, wherein the securing element is formed at least partially from a plastic, a composite material, or a soft foam.

3. The drug delivery device of claim 1, wherein the drug-releasing element comprises a combination of at least two anti-epileptic substances.

4. The drug delivery device of claim 1, wherein the securing element further comprises an additional securing aid.

5. The drug delivery device of claim 4, wherein the securing aid is selected from the group consisting of ratchet-like features, serrations, suction cups, adhesive materials, a tether, and combinations thereof.

6. The drug delivery device of claim 1, further comprising a syringe feature attached to the drug-releasing element.

7. The drug delivery device of claim 1, further comprising at least one magnet mounted onto the securing element.

8. The drug delivery device of claim 1, further comprising a voltage source capable of applying a voltage between the drug-releasing element and the securing element via an electrical conducting material.

9. The drug-delivery device of claim 8, wherein the drug-releasing element and securing element are electrically polarized.

10. A drug delivery device comprising:
a securing element having a first portion, a second portion, and a third portion;
a drug-releasing element connected to the first portion of the securing element, wherein the drug-releasing element comprises at least one drug or drug-containing substance, and wherein the drug-releasing element is capable of releasing the at least one drug or drug-containing substance in the oral cavity.

11. The drug delivery device of claim 10, wherein the at least one drug-containing substance is in the form of a pill, tablet, or capsule.

12. The drug delivery device of claim 10, wherein the at least one drug-containing substance comprises an outer rapid drug-releasing film and an inner slower drug-releasing matrix.

13. The drug delivery device of claim 10, wherein the at least one drug-containing substance comprises at least one anti-epileptic substance.

14. The drug delivery device of claim 10, further comprising a hinge feature disposed between the first portion and the second portion of the securing element.

15. The drug delivery device of claim 10, further comprising a handle attached to the second portion of the securing element.

16. A drug delivery device comprising:
a securing element having a first portion, a transitional portion, and a third portion, wherein the first portion is capable of being positioned inside the oral cavity of a patient, the transitional portion is capable of traversing the lips of the patient, and the third portion is capable of being positioned against the cheek of the patient; and
a drug-releasing element connected to the first portion of the securing element, wherein the drug-releasing element comprises at least one drug, and wherein the drug-releasing element further comprises at least one microneedle capable of delivering the at least one drug through contact of the oral cavity.

17. The drug delivery device of claim 16, wherein the at least one drug is coated on an outside surface of the microneedle.

18. The drug delivery device of claim 16, wherein the at least one drug is positioned in an internal cavity of the microneedle.

19. The drug delivery device of claim 16, wherein the microneedle is made of non-dissolvable material.

20. The drug delivery device of claim 16, wherein the microneedle is made of dissolvable material.

21. The drug delivery device of claim 16, wherein the length of the microneedle is about 0.10 mm to about 0.75 mm.

22. The drug delivery device of claim 16, further comprising an identification element attached to the securing element.

23. The drug delivery device of claim 16, wherein the drug-releasing element is connected to the first portion of the securing element by a connecting segment.

24. The drug delivery device of claim 16, wherein the patient is a human.
